(19) United States
(12) Patent Application Publication
(10) Pub. No.: US 2013/0150905 A1
(43) Pub. Date: Jun. 13, 2013

(54) Rod Holder

(71) Applicant: Edward Karpowicz, Sewell, NJ (US)

(72) Inventor: Edward Karpowicz, Sewell, NJ (US)

(21) Appl. No.: 13/315,749

(22) Filed: Dec. 9, 2011

(51) Int. Cl. A61B 17/00; B23P 11/00

(52) U.S. Cl. 606/86 A; 29/428; 29/525.11

(57) ABSTRACT

A rod holder for inserting a rod for spinal instrumentation into a patient. The rod may be inserted through the same incision used for fixing pedicle screws, so that an additional incision may not be required. The rod holder may include a body, a slide that slidably mates to a side of the body, and a ram that actuates the slide.

### BACKGROUND OF THE PRESENT DISCLOSURE

1. Field of the Present Disclosure

The present disclosure is directed to a device and a method for inserting a rigid rod into a patient who is undergoing spinal instrumentation.

2. Related Art

Spinal instrumentation may be used to treat a variety of conditions, including injury and deformity due to trauma or congenital defect. Spinal instrumentation may also be used in a spinal fusion procedure to alleviate the difficulties caused by a damaged or herniated intervertebral disc. Spinal instrumentation typically includes a number of bone screws for attaching to individual vertebrae. The pedicle is a preferred attachment point because it is easily accessible from the back of the patient and it offers a great depth of cortical bone. Once the pedicle screws are in place, one or more rods are placed substantially parallel to the long axis of the spine and connected to the pedicle screws. Each rod is usually connected to at least two screws, each of which is attached to a different vertebra. The entire construct serves to properly align and/or separate the vertebrae. It may be supplemented with bone grafts, bone cement, or the like to promote healing, long-term stability, and so on.

Traditionally, spinal instrumentation was installed via open-back surgery. This type of procedure causes extensive trauma to the patient, resulting in long and painful recovery times. In recent years, a shift has been made toward minimally invasive surgery techniques. In minimally invasive surgery, the surgeon makes only one to a few small incisions and uses special tools to insert devices, observe his progress, and perform other activities in the surgical site. The result is much less injury to the patient and improved healing and recovery times.

The pedicle screws commonly used in instrumentation procedures utilize an open-head design to connect to the rods. This head has a flat base with an arm rising on either side, giving it a U-shaped profile. The rod is placed between the upright portions of the open head, and a locking clamp, which connects to the uprights, is placed over the rod to lock it in place. Closed-head screws, which have an integral locking mechanism for securing the screw to the rod, are also common. A sleeve may be connected to the uprights of each screw and used to guide tools and components onto the pedicle screws.

Once the screws are in place, a specialized tool, known as a rod holder, is used to insert the rod. Current rod holders require the surgeon to make an additional incision, beyond those used to insert and secure the pedicle screws, to insert the rod. For the patient, the additional incision represents further trauma, longer recovery time, and increased pain. The patient could avoid these added complications if the added incision could be eliminated.

Accordingly, there is a need for a rod holder that can be used to insert a rod through the same incision used to insert the pedicle screws.

### SUMMARY OF THE PRESENT DISCLOSURE

The present disclosure meets the foregoing need and allows a rod to be inserted into a patient without requiring an additional incision, which results in a significant reduction in trauma and recovery time, as well as other advantages apparent from the discussion herein.

Accordingly, one aspect of the present disclosure describes a rod holder that includes a body, a slide, and a ram. The slide is structured and arranged to slidably mate to a side of the body. The ram is connected to the body and the slide. In addition, the ram is structured and arranged to actuate the slide along a side of the body. The slide includes an open position and a closed position.

The rod holder may include a handle. The body may include a rail and the slide may include a slot structured and arranged to receive the rail. The rail may include a tabbed portion and a non-tabbed portion. The slot may include a narrower portion and a wider portion. The narrower portion may be structured and arranged to interact with the tabbed portion to slidably mate the slide to the body when the slide is in an open position or a closed position. The slide may include a tab, and the body may include a slot that is structured to receive the tab on the slide. The tab and the slot may form a hole that is structured to retain a rod when the slide is in a closed position. The ram may include a nut and a positive stop. The ram may include a knob, and the slide may include a slot structured to receive the knob.

According to another aspect of the present disclosure, a method of assembling a rod holder includes providing a body, threading a ram through a hole in the body, and connecting a slide to the body and the ram. The method may include attaching a handle to the body. The method may also include attaching a nut to the ram.

In yet another aspect of the present disclosure, a method of using a rod holder includes providing a rod holder that includes a body and a ram threaded through a hole in the body. A slide is mated to the body and the ram so that the rain is able to actuate the slide along a side of the body. A rod is placed through an opening in the body, and the slide is actuated until the rod is retained by the slide and the body. The rod is threaded through at least one sleeve that has been inserted through an incision into a patient and connected to a bone screw that is attached to a bone in the patient. The rod is inserted into the patient with the rod holder, and the rod holder enters the same incision used by the sleeve(s).

The rod holder may include a handle connected to the body. The rod may be threaded through two sleeves, each connected to a different bone screw. Rotating the ram may actuate the slide.

Additional features, advantages, and aspects of the present disclosure may be set forth or apparent from consideration of the following detailed description, drawings, and claims. Moreover, it is to be understood that both the foregoing summary of the present disclosure and the following detailed description are exemplary and intended to provide further explanation without limiting the scope of the present disclosure as claimed.

### BRIEF DESCRIPTION OF THE DRAWINGS

The accompanying drawings, which are included to provide a further understanding of the present disclosure, are incorporated in and constitute a part of this specification, illustrate aspects of the present disclosure and together with the detailed description serve to explain the principles of the present disclosure. No attempt is made to show structural details of the present disclosure in more detail than may be necessary for a fundamental understanding of the present disclosure and the various ways in which it may be practiced. In the drawings:

FIG. 1A shows a rod holder according to an aspect of the present disclosure;

FIG. 1B shows the rod holder holding a rod;

FIG. 2 shows an exploded view of the rod holder;

FIG. 3 shows the rod holder with the top of the slide exposed;

FIG. 4A shows the rod holder in a pre-assembly arrangement;

FIG. 4B shows the rod holder in a fully open configuration;

FIG. 4C shows the rod holder in an open position;

FIG. 4D shows the rod holder in a closed position;

FIG. 5A illustrates one way the rod holder may be used with sleeves, pedicle screws, and a rod; and

FIG. 5B shows a perspective view of the holder in place with pedicle screws, sleeves, and a rod.

### DETAILED DESCRIPTION OF THE PRESENT DISCLOSURE

The aspects of the present disclosure and the various features and advantageous details thereof are explained more fully with reference to the non-limiting aspects and examples that are described and/or illustrated in the accompanying drawings and detailed in the following description. It should be noted that the features illustrated in the drawings are not necessarily drawn to scale, and features of one aspect may be employed with other aspects as the skilled artisan would recognize, even if not explicitly stated herein. Descriptions of well-known components and processing techniques may be omitted so as to not unnecessarily obscure the aspects of the present disclosure. The examples used herein are intended merely to facilitate an understanding of ways in which the present disclosure may be practiced and to further enable those of skill in the art to practice the aspects of the present disclosure. Accordingly, the examples and aspects herein should not be construed as limiting the scope of the present disclosure, which is defined solely by the appended claims and applicable law. Moreover, it is noted that like reference numerals represent similar parts throughout the several views of the drawings.

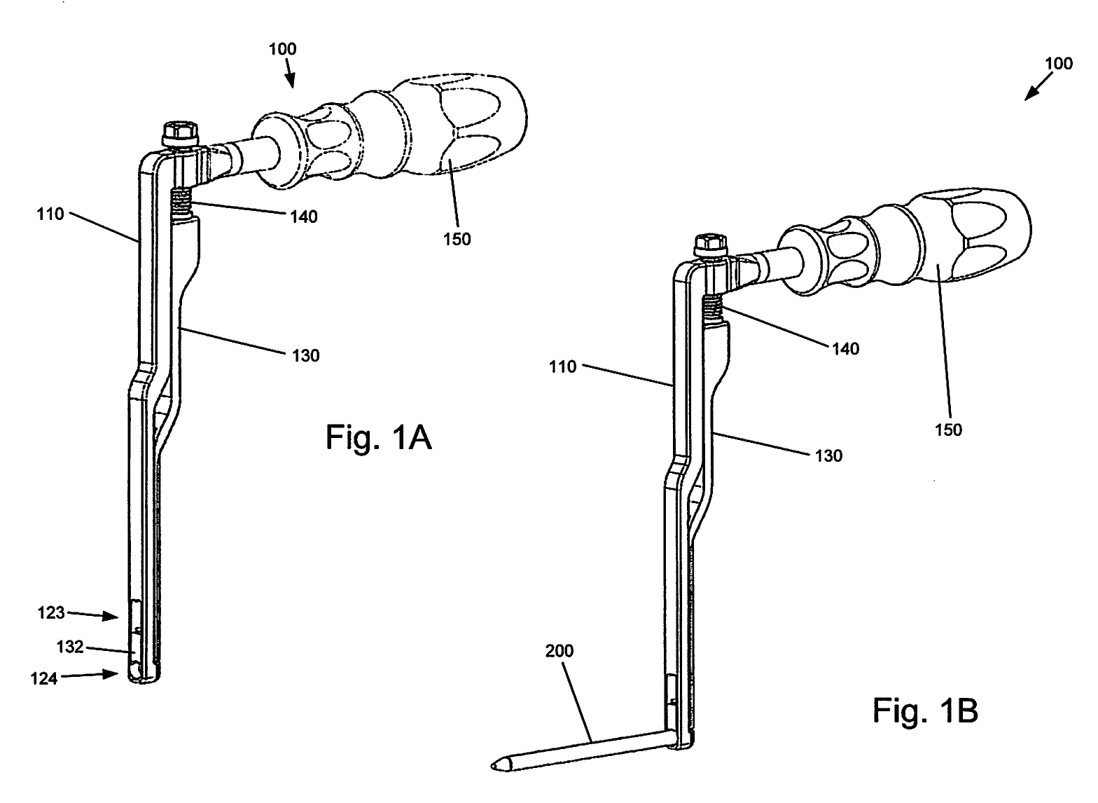

FIG. 1A shows a rod holder 100 according to an aspect of the present disclosure, and FIG. 1B shows the rod holder 100 holding a rod 200. The rod holder 100 may include a body 110, a handle 150, a slide 130, and a ram 140. By twisting the ram 140, a surgeon or other user of the rod holder 100 may actuate the slide 130 along the length of the body 110 to grip and release a rod 200. The slide 130 may mate to a side of the body 110. A tab 132 on the slide 130 may extend through a slot 123 in the body. The tab 132 and the slot 123 may form a hole 124 for holding, gripping, and/or retaining the rod 200.

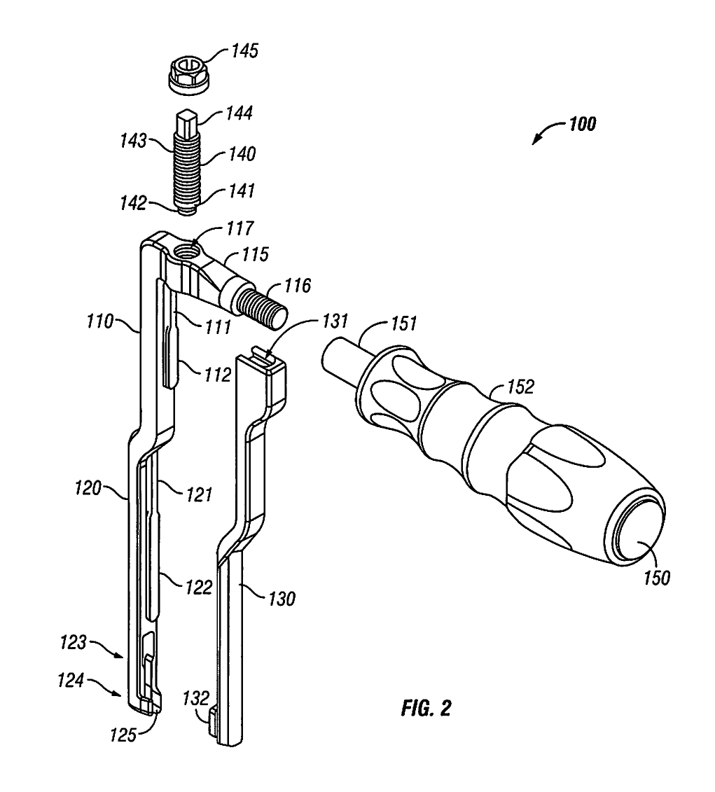

FIG. 2 shows an exploded view of the rod holder 100. The body 110 may include a portion 115 set at approximately a right-angle to the main body 110. The right-angle portion 115 may include a threaded shaft 116 for connecting to the handle 150. The right-angle portion may also include a threaded hole 117 for receiving the ram 140.

The body may include an extension 120, which may be offset from the main body 110 but substantially parallel to it. The extension 120 may be of a narrower or lighter weight construction than the main body 110. The side of the body 110 under the right-angle portion 115 may include a first rail 111 and a second rail 121. Both rails 111, 121 may have tabbed portions 112, 122. The remainder of each rail 111, 121 may be regarded as a non-tabbed portion. The slide 130 may actuate along the rails 111, 121, as described below. The distal end of the body 110 may include a slot 123 for receiving the tab 132 of the slide. The distal end of the body 110 may further include a tab 125 that may act as a stop for the slide 130. When the slide 130 abuts the tab 125, the two components may form a hole 124 for holding a rod 200 (seen in, e.g., FIG. 1B).

The slide 130 may have a shape that matches or conforms to the side of the body 110. A tab 132 may be located at the lower or distal end of the slide 130, on the side that faces the body 110. The upper or proximal end of the slide 130 may have a slot 131 for interfacing with a knob 142 on the ram 140, as described below.

The ram 140 may include a threaded shaft 143 and a nut 145. The shaft 143 may be inserted into and threaded through the threaded hole 117 from the bottom or distal side. The shaft may include a positive stop 141, which may be wider than the hole 117. The positive stop 141 may prevent the ram 140 from being threaded completely through the hole 117. The distal end of the ram 140 may include a knob 142, which may have a wider head and a narrower neck. The neck may be narrow enough to fit through the slot 131 on the slide 130, while the head of the knob 142 may be too wide to fit through the slot 131. By sliding or fitting the slide 130, including slot 131, over the knob 142, the slide 130 may be held or retained by the ram 140. The positive stop 141 and the knob 142 may work together to allow easy and repeatable attachment and removal of the slide 130 for cleaning and/or sterilization.

The proximal end of the threaded shaft 143 may include a receiver 144 for a nut 145. The receiver 144 may have a cross-section that is square, pentagonal, hexagonal, or any other appropriate shape. The nut 145 may include a hole that may include a complementary shape for mating with the receiver 144. The hole may be lined with nylon or another material that may deform elastically to reduce or substantially prevent the nut from loosening or detaching from the receiver 144. The nut 145 may be a wing nut or include other features to allow easy and convenient rotation and/or actuation of the ram 140.

The nut 145, which may be located on the proximal end of the ram 140, and the positive stop 141, which may be located near the distal end of the ram 140, may work together to retain the ram 140 within the hole 117. For example, the nut 145 may prevent the ram 140 from being threaded too far distally through the hole 117, and the stop 141 may prevent the ram 140 from being threaded too far proximally. This arrangement may make the ram 140 fully captured by the body 110 of the holder 100, thereby preventing accidental unthreading.

The handle 150 may include a grip 152 and a shaft 151. The shaft 151 may be internally threaded to mate with the externally threaded shaft 116 on the body 110. The grip 152 may be structured and contoured to provide a comfortable and ergonomic hand position to a surgeon or other user who may be holding the holder 100 in either hand.

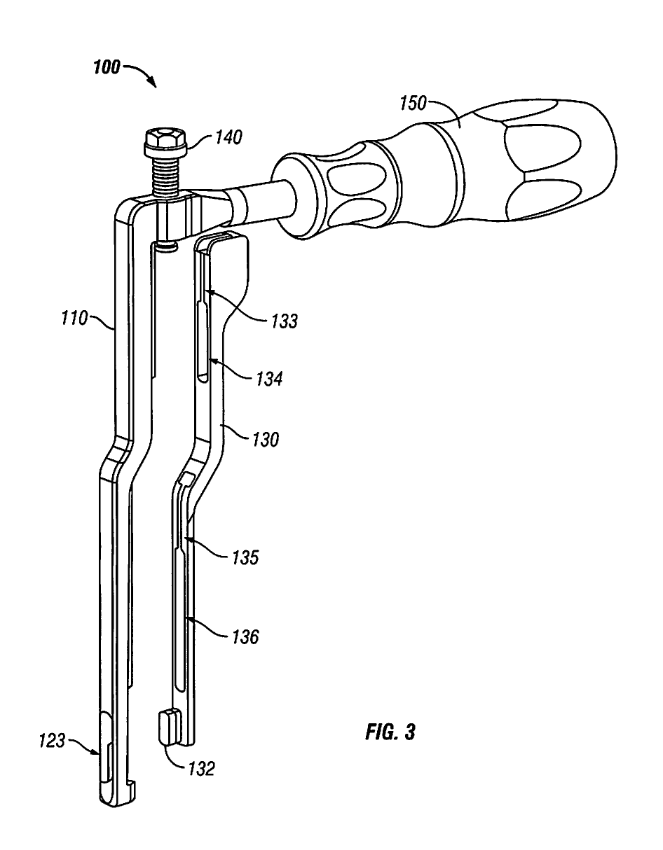

FIG. 3 shows the rod holder 100 with the top of the slide 130 exposed. The top of the slide may include a slot 133 with a wider portion 134, as well as a second slot 135 with a wider portion 136. The remainder of the slots 133, 135 may be regarded as narrower portions. When the slide 130 is mated to the body 110, the slot 131 may fit over the knob 142 on the distal end of the ram 140. This interaction may allow the ram 140 to retain the slide 130 and to actuate it, as described below. The slot 133 on the slide 130 may mate to the rail 111 on the body. The wider portion 134 of the slot 133 may fit over the tabbed portion 112 of the rail 111, and the narrower portion of the slot 133 may fit over the non-tabbed portion of the rail 111. Similarly, the slot 135 may mate to the rail 121. The wider portion 136 of the slot 135 may fit over the tabbed portion 122 of the rail 121, and the narrower portion of the slot 135 may fit over the non-tabbed portion of the rail 121. In addition, the tab 132 may have a cross-section similar to that of the tabbed portion 122 of the rail 121. That is, the tab 132 may be wider on top and have a narrower neck that connects the wider portion to the main structure of the slide 130. The tab 132 may be inserted into the proximal portion of the slot 123. The distal portion of the slot 123 may have rails that are complementary to the neck of the tab 132.

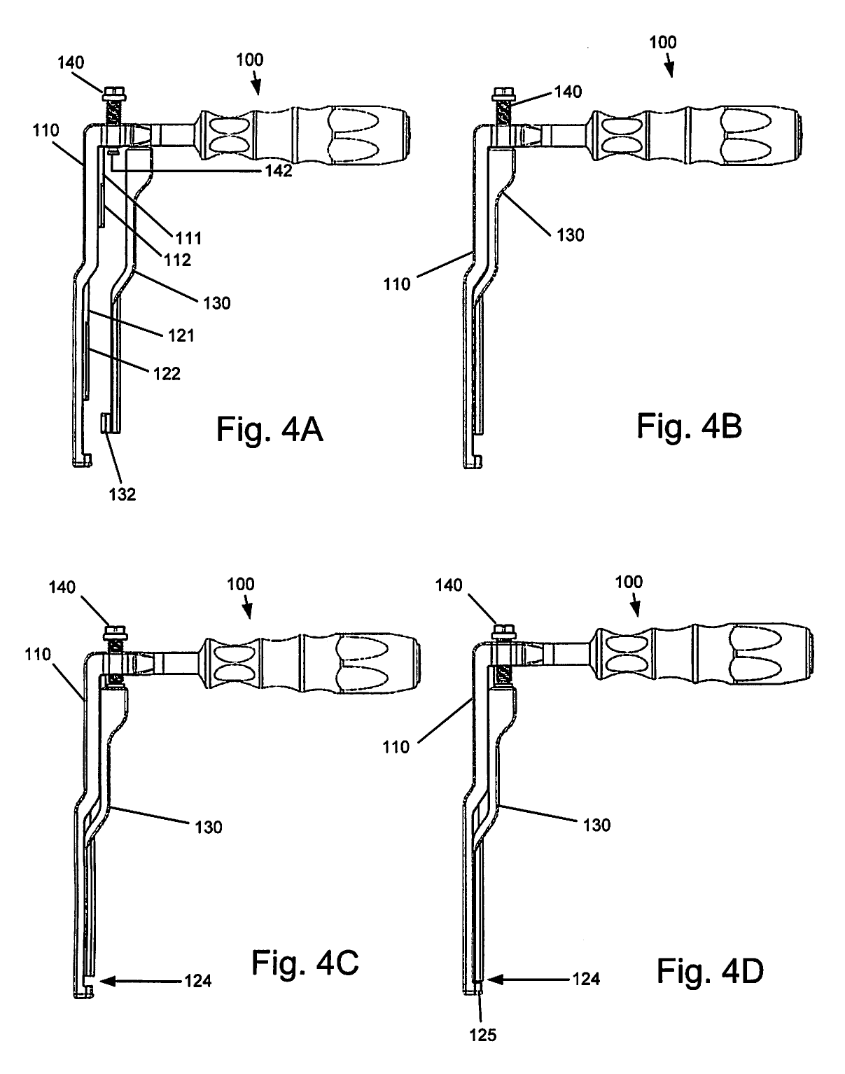

FIG. 4A shows the rod holder 100 in a pre-assembly arrangement or configuration. The ram 140 may be actuated to its most proximal position. This may cause the knob 142 to align with the slot 131 in the slide 130 when the slide abuts the right-angle portion 115 of the body 110, as seen in FIG. 4A. This positioning may also align the tabbed portion 112 and the non-tabbed portion of the rail 111 and with the wider portion 134 and the narrower portion of the slot 133, respectively. Similarly, the tabbed portion 122 and the non-tabbed portion of the rail 121 and may be aligned with the wider portion 136 and the narrower portion of the slot 135, respectively. In addition, the tab 132 may be aligned with the proximal portion of the slot 123.

By moving the slide 130 until it abuts the body 110, a user of the rod holder may assemble the device, resulting in the fully open configuration seen in FIG. 4B. The matings between the complementary components, described above, may be complete. Removing the slide 130 from the body 110 may be just as simple. By twisting the nut 145 on the ram 140, a user of the rod holder 100 may actuate the slide in a distal direction, which may result in the configuration seen in FIG. 4C.

FIG. 4C shows the rod holder 100 in an open position, ready to accept a rod. The ram may be actuated, moving it distally. The slide may be displaced in a distal direction, as a result. This displacement may cause the narrower portion of the slot 133 to overlap with the tabbed portion 112 of the rail 111, which may retain the slide 130 against the body 110 as shown in FIG. 4C. In addition, the narrower portion of the slot 135 may overlap with the tabbed portion 122 of the rail 121. Further, the tab 132 may be retained by the rails located in the distal portion of the slot 123. A rod 200 (seen in FIG. 1B) may be inserted into the hole 124 and the ram may be actuated distally to move the slide 130 until it contacts the tab 125.

FIG. 4D shows the rod holder 100 in a closed position. A rod 200, which may be inserted when the holder 100 is in the open position seen in FIG. 4C, may be retained in the hole 124 for inserting into a patient. The ram 140 may be actuated to (or near) its most distal position, which may be limited by the nut 145. The tab 125 may also limit the distal motion of the ram 140 via the connection of the ram 140 to the slide 130. When the rod 200 is properly positioned, e.g., in a patient, it may be released from the holder 100 by actuating the ram 140 in a proximal direction and reversing the sequence described herein. Once the holder 100 is empty, it may be loaded with an additional rod 200 for insertion into the patient. Once the operation is complete, the slide 130 may be separated from the body for proper cleaning and sterilization.

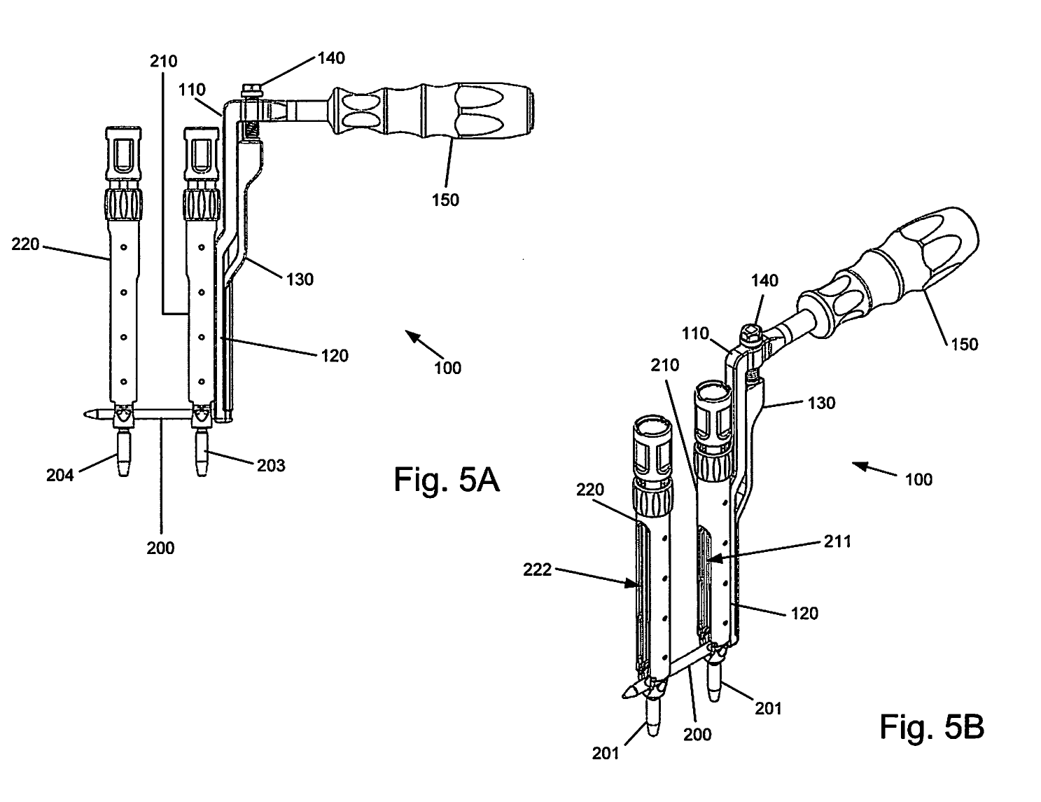

FIG. 5A illustrates an example in which the rod holder 100 may be used with other common implements for spinal surgery, such as, e.g., sleeves 210, 220; pedicle screws 201; and the rod 200. FIG. 5A shows a bi-level procedure, i.e. joining two vertebrae, but the rod holder 100 may be used with procedures that involve any number of vertebrae. In this procedure, an incision may be made in the patient's back. Next, the sleeve 210 may be guided into the incision and into contact with one vertebra. Then the second sleeve 220 may be inserted into the same incision and guided into contact with an adjacent vertebra. The sleeves 210, 220 may create relatively clear paths into the body and allow the surgeon to insert one pedicle screw 203, 204 into each of the vertebrae. The pedicle screws 201 may have an open head design. The open head design may include a slot along the middle for receiving the rod 200 and uprights on either side of the slot for connecting to the respective sleeve 210, 220 and retaining the rod 200.

Once the rod 200 is in position in the holder 100, it may be inserted through each of the sleeves 210, 220 and lowered into the incision. Because of its small cross-section and narrower design, especially in the extension portion 120 of the body 110, the holder 100 may be inserted into the same incision that is occupied by the sleeves. Previous designs required an additional incision to place the rod 200 in the screw heads. After the rod 200 is in place in the screw heads, it may be locked in place by the used of locking caps that connect to the uprights on each screw head. Then the ram 140 may be reversed, thereby releasing the rod 100 from the grip of the holder 100.

FIG. 5B shows a perspective view of the holder 100 in place with the pedicle screws 201; the sleeves 210, 220; and the rod 200. A larger opening 222 may be seen on the sleeve 220, and a smaller opening 111 may be seen on the sleeve 210. Note that, as seen in FIGS. 5A and 5B, the sleeve 210 is identical to the sleeve 220, rotated about 180° . Thus, the sleeve 220 may have a smaller opening 221 that may be facing the sleeve 210, and the sleeve 210 may have a larger opening 212 facing the body 110 of the holder 100. This series of openings may assist with inserting the rod 200 into the patient and affixing it to the pedicle screws 201.

When inserting the rod 200 into the pedicle screws 203, 204 with the holder 100, for example, the surgeon may first insert the rod 200 into the larger opening 212 of the sleeve 210, pass it through the sleeve 210, and bring it out through the smaller opening 211. The rod 200 may then continue into the smaller opening 221 of the sleeve 220 and emerge from its larger opening 222. This may be done above level of (or away from) the patient's skin so that, e.g., the rod is properly positioned and aligned before being inserted. The surgeon may then use the holder 100 to pass the rod 200 into the incision, with the holder 100 itself entering into the same incision used by the sleeves 210, 220. The openings 211, 212, 221, 222 in the sleeves 210, 200 may guide the rod 200 into the heads of the pedicle screws 201. The sleeves may also guide the locking caps (not shown) onto the rod 200 and the screw heads. While the above description may encompass open-head pedicle screws, the rod holder 100 may also be utilized or operated in conjunction with closed-head pedicle screws and other types of bone screws, as will be understood by those skilled in the art.

While the present disclosure has been described in terms of exemplary aspects, those skilled in the art will recognize that the present disclosure can be practiced with modifications in the spirit and scope of the appended claims. These examples given above are merely illustrative and are not meant to be an exhaustive list of all possible designs, aspects, applications or modifications of the present disclosure.

1. A rod holder comprising:
a body;
a handle connected to the body;
a slide configured to slidably mate to a side of the body; and
a ram connected to the body and the slide, the ram configured to actuate the slide along side of the body between an open position and a closed position,
wherein the handle is disposed generally perpendicular to the body.

2. The rod holder of claim 1, wherein the handle is threadably engaged to the body.

3. The rod holder of claim 1, wherein:
the body comprises a rail; and
the slide comprises a slot configured to receive the rail.

4. The rod holder of claim 3, wherein:
the rail comprises a tabbed portion and a non-tabbed portion; and
the slot comprises a narrower portion and a wider portion, the narrower portion configured to interact with the tabbed portion to slidably mate the slide to the body.

5. The rod holder of claim 1, wherein:
the slide comprises a tab; and
the body comprises a slot configured to receive the tab, the tab and the slot forming a hole configured to retain a rod when the slide is in a closed position.

6. The rod holder of claim 1, wherein the ram comprises a nut and a positive stop.

7. The rod holder of claim 1, wherein:
the ram comprises a knob; and
the slide comprises a slot configured to receive the knob.

8. A method for assembling a rod holder, the method comprising:
providing a body;
threading a ram through a hole in the body; and
connecting a slide to the body and to the ram.

9. The method of claim 8, further comprising attaching a handle to the body.

10. The method of claim 8, further comprising attaching a nut to the ram.

11. A method of using a rod holder, the method comprising:
providing a rod holder, the rod holder comprising a body, a handle connected to the body., a slide, and a ram threaded through a hole in the body, the slide being mated to the body and the ram so that the ram can actuate the slide along a side of the body, the handle being connected generally perpendicular to the body;
placing a rod through an opening in the body;
actuating the slide until the rod is retained by the body and the slide;
threading the rod through at least one sleeve, the sleeve having been inserted through an incision into a patient, the sleeve connected to a bone screw attached to a bone in the patient; and
inserting the rod into the patient using the rod holder, the rod holder entering the patient through the same incision used by the sleeve.

12. The method of claim 11, wherein the handle is threadably connected to the body.

13. The method of claim 11, wherein the at least one sleeve comprises two sleeves, each connected to a different bone screw.

14. The method of claim 11, wherein actuating the slide comprises rotating the ram.
